(19) United States
(12) Patent Application Publication
(10) Pub. No.: US 2010/0286571 A1
(43) Pub. Date: Nov. 11, 2010

(54) System and Method for Providing Body Sway Feedback to a Body of a Subject

(76) Inventors: John H. J. Allum, Iseltwald (CH); Juergen Burger, Biel (CH); Daniel Debrunner, Inkwil (CH); Simon Meyes, Munchenbuchsee (CH); Roger Tschanz, Uetendorf (CH)

Correspondence Address: MAYBACK & HOFFMAN, P.A., 5722 S. FLAMINGO ROAD #232, FORT LAUDERDALE, FL, 33330, US

(21) Appl. No.: 12/514,938

(22) Filed: May 14, 2009

Related U.S. Application Data
(63) Continuation of application No. PCT/EP2006/011080, filed on Nov. 17, 2006.

(51) Int. Cl. A61B 5/11; A61H 1/00

(52) U.S. Cl. 600/595; 601/46

(57) ABSTRACT

The present invention relates to a system (10) and to a method for providing body sway feedback to a body of a subject. The system (10) comprises (a) at least one body sway sensor means (12) adapted to be attached the body and adapted for providing body sway signals indicative of a sway of the body, (b) a system processor means (14) adapted for deriving body sway information from the body sway signals and adapted for generating a body sway feedback signal from the body sway information, and (c) a subject body sway feedback means (40) having at least one transducer unit (42, 44) and adapted for providing a body sway feedback to the body via said at least one transducer unit (42, 44) based on said body sway feedback signal, (d) wherein said at least one transducer unit (42, 44) is or comprises at least one transducer, (e) wherein said at least one transducer is adapted to be contacted to a head of the body, adapted for vibrating, and adapted for transferring a respective vibration to the head, and (f) wherein said at least one transducer is adapted to evoke based on the vibration a multimodal body sway feedback as stimuli to the subject having a plurality of kinds of stimuli of the group comprising auditory stimuli, tactile stimuli, and vestibular stimuli.

### FIELD OF INVENTION

The present invention relates to a system and a method for providing body sway feedback to a body of a subject.

The present invention—inter alia—pertains generally to methods and devices for providing non-invasive feedback of postural sway to a human subject during standing or movement tasks, and more particularly to such methods and devices that employ direct measurement of body position using displacement or motion transducers or other sensing devices attached to the body to provide a feedback of body sway to the subject. This invention pertains specifically to methods and devices that provide multi-sensory modes of the feedback of the measured postural information to the subject using transducers attached to the head which are capable of operating in all or at least two of the following sensory modes simultaneously: bone-conducting aural, vibro-tactile, vibro-vestibular, and, if desired a visual mode.

### BACKGROUND OF THE INVENTION

Individuals who suffer from a balance control deficit are abnormally prone to falling and have poor gait control when walking or engaging in other movement tasks. A balance control deficit may be the result of a wide variety of sensory and/or motor disorders that impair the posture and equilibrium control of the subject. Such an impairment may also be caused by decreased sensory and motor function with aging. In order to make a correct assessment of a subject's balance deficit, and thereby to take remedial measures, an examining physician or physical therapist, must determine the subject's balance control abilities for a number of motor tasks, such as standing with eyes closed, walking while moving the head, getting up out of a chair, walking over low barriers, etc. By observing the subject performing such motor tasks, the physician may be able to determine if the subject's balance control is within normal limits for his age grouping. However, to provide a more accurate and objective assessment of the individual's balance control deficit a test system measuring body sway is required.

After a balance deficit has been quantified, but possibly not completely diagnosed, a physician may prescribe remedial measures to help bring the subject's balance control nearer to or within normal limits. The physician may prescribe medication for several weeks that alters the effect of peripheral balance senses on the brain. Alternatively the physician may prescribe a course of physical therapy, which will last several weeks, with the aim of training the subject's brain to deal with a reduced sense of balance when trying to maintain the body upright and prevent a fall. However, neither of these techniques will have an immediate rehabilitatory effect on the subject's balance deficit. Moreover, medication can have side effects, and also reduce the processing capabilities of the brain. A course of physical therapy requires a long training period extending over 2 months. These difficulties and limitations associated with conventional remedial measures for dealing with balance deficits are most problematic when the subject is older than 65 years of age and likely to have a falling tendency.

One manner to overcome the aforementioned difficulties and limitations might be to employ a balance prothesis which could provide an addition to the sense of balance registered by the brain from a variety of sensory sources including a major input from the vestibular system in the inner ear, from proprioceptive receptors, especially those in muscles of the ankle joint, and the visual system. In the field of hearing which is physiologically related to that of balance, prostheses are routinely used to augment a subject's hearing ability by electrically stimulating the acoustic nerve through per- or trans-cutaneous means. Some prosthetic devices have been described to use the same means to stimulate the vestibular nerve, however their use is currently limited to animal studies as efficacy in animals, a necessary step before use in man, has not been demonstrated to date. While some prosthetic devices have been described using vibro-tactile stimulation these have the disadvantages and limitations that the sensory information is applied to the body far from the head and therefore has a long transmission time to the brain. Such a system has been proposed for use around the waist. Not only is its point of application far from the CNS but it may also cause blocking of proprioceptive signals from nearby trunk muscles due to the vibration of the vibro-tactile transducers. Another disadvantage of previously described systems is that feedback information is limited to one sensory mode per prosthesis system, such as an air-conducted acoustic feedback. Because a subject's perception, for example hearing, may be limited in the chosen sensory feedback mode, such feedback may be likewise limited in use.

The current invention overcomes these disadvantages and limitations by using transducers which, being mounted on the head, can be multi-modal, offering bone-conducted acoustic feedback, vibro-tactile feedback and vibro-vestibular feedback, as well as having a short transmission time to the brain.

Furthermore the bone-conducted acoustic feedback overcomes the disadvantage of air-conducted acoustic feedback in that it is hardly audible to others near the subject who is receiving the feedback and also leaves open the channel of communication with the subject via air-conducted speech or other sounds. The present invention overcomes these disadvantages because it uses vibro-tactile vibration at the skull which will also excite the auditory and vestibular receptors in the inner ear. Such a system and method offer advantages in that it is multi-modal and has short transmission terms to the brain.

### SUMMARY OF INVENTION

It is an object underlying the present invention to provide a system/apparatus and a method for providing a body sway feedback to the body of a subject in a more reliable manner.

The object underlying the present invention is solved by a system/apparatus and a method for providing a body sway feedback to the body of a subject according to independent claims 1 and 20.

The present invention inter alia provides a system/apparatus and a method for providing a multi-modal feedback information to a human subject of the subject's postural sway during standing or movement tasks. A method or device in accordance with the present invention may be used as a rehabilitory tool for subjects prone to abnormal falling or who wishes to improve their movement control. The present invention may be used to provide prosthetic feedback to aid in the rehabilitation of balance and gait disorders without interfering with natural sensory signals. Because the prosthetic feedback is applied at the head in the form of a vibration it creates in the skull bone-conducted auditory signals. Thus the interference with other, normally air-conducted, auditory signals is minimized. Furthermore the use of vibration at the skull, above the level of the ears also excites vibro-tactile skin receptors. The interference of such signals with muscle proprioceptive signals when such a vibro-tactile signal is applied near or on a muscle will not occur. Finally the application of vibration at the head permits the excitation of vestibular receptors (vibro-vestibular) in the head using such signals.

The method and the apparatus or system of the present invention are based on the finding that unitary sense of balance computed by the human brain involves monitoring the upper body and maintaining the angular position and angular velocity of the body within a cone of angular stability. For this purpose the brain depends primarily on proprioceptive signals, which are generated some distance away from brain, as well as vestibular and visual signals. Once the cone of angular stability (the cone of stability is defined by the amount a subject can lean in an erect posture in each direction—front, back, left and right—without falling) is exceeded, with an excessive angular velocity, normal individuals will respond by correcting the trunk position within 200 ms. If however natural balance signals are deficient, or slow in reaching the brain, balance will not be corrected rapidly enough. The current invention provides multi-modal feedback rapidly to the brain by being applied at the head and supplements natural feedback signals. It is based on our finding that vibratory feedback applied to the skull is perceived via acoustic, vibro-tactile and vibro-vestibular sensory systems.

The present invention employs wearable body sway sensors, such as angular velocity sensors, as those that are preferable attached to the upper body at the level of the lower back. The sensor output signals are transformed into detailed angular displacement and velocity information by a microprocessor based system processor. The system processor is programmed to transform the angular displacement and velocity information into useful information formats that are displayed to an operator on an operator's display unit. Quantified body sway information that may be provided to the operator as part of an operator's display includes: time histories of the subject's angular sway deviations in the pitch and roll directions, x-y plots of the subject's angular sway deviations in the pitch and roll directions, x-y plots of the subject's angular velocity deviations, where x is the roll deviation and y the pitch deviation at an instant of time, areas of such x-y plots, histograms of the angular sway deviations and angular velocities in the pitch and roll directions, measures of the peak-to-peak deviations of angular sway and angular velocity in the pitch and roll directions. Apart from comparisons to thresholds and limits of each feedback mode the operator's display may also provide for comparisons between examination trial results of the same type of examination and a summary of results from different types of examinations with and without the feedback of this invention and comparisons to body sway information obtained from a normal population of approximately the same age as the test subject. Since neither the body sway sensors nor the prosthetic feedback device employed by the current invention interfere with subject movement, this detailed information then may be obtained for examination trials involving the performance of a wide variety of movement tasks by the subject, such as walking up and down stairs, walking over barriers, or simple stance tasks.

The system processor of the present invention is normally programmed to provide rehabilitory postural feedback to subject based upon the body sway angle and sway velocity information obtained from the body sway sensors. This feedback is applied at head and may be in the form of multi-modal bone-conducted auditory, vibro-tactile, bone-conducted vestibular feedback and, in addition, if desired visual stimulation. Depending on the placement or intensity of the feedback stimulation, a single type of feedback may be present or all three different types of feedback (bone-conducted auditory, vibro-tactile or vibro-vestibular) may be provided to the subject in combination. The rehabilitory feedback immediately augments the balance signals normally used by the subject's brain to help stabilize body sway and improve balance. The feedback threshold for each of the 3 modes (bone-conducted auditory, vibro-tactile or vibro-vestibular) and the feedback thresholds for visual feedback, that is the amplitude of body sway angle and/or angular velocity when the feedback is first active, the amount of feedback relative to the body sway angle and/or angular velocity, the upper limit of feedback modulation, and the sway directions over which feedback could be disabled, may be adjusted by the system operator. The information provided in the operator's display provides an objective measure for determining improvements in balance brought about by the application of feedback and by altering feedback thresholds, gains, limits, and active feedback directions.

For the auditory feedback mode of the multi-modal feedback, information on the amount of angular sway and/or angular velocity of sway is presented to the subject aurally in the form of bone-conducted sounds transmitted via one or more vibrators which act as bone-conducting acoustic transducers. To be close to the inner ear these are placed on the skull over the left ear or over the right ear or over both ears. Three different audible centre frequencies, one for forward sway displacement, one for backward displacement and one each for left and right sway displacements are generally presented at each left and right transducer, respectively. The volume of these tones increases according to the feedback gain as the subject sways in the pitch and roll directions or combinations thereof. To make the tones for left and right displacement more noticeable the tone frequencies are shifted slightly in frequency every ca. 20 ms. The sway angle at which the tone is first heard, the depth of tone volume modulation with increasing sway angle, and the angle at which the tone volume increase is limited, as well as if feedback for sway in some directions is absent, are auditory feedback parameters, which may be set by the operator to help improve the subject's control of body sway, and therefore improve the subject's balance control for one or more movement tasks.

Vibro-tactile feedback via skin receptors can also be provided by the same or different vibrators mounted on a headband used to convey an acoustic signal to the ear. These vibro-tactile signals can be used to convey a sense of body sway. By placing several vibrators at different locations on the headband with spacing according to the sensitivity of the skin of the head, that is, more vibrators at the back of the skull where tactile sensitivity of the skin is greater than at the front, and setting each vibrator on when angular sway of the body is in the direction of the vibrator, a sense of body sway in that direction may be perceived by the subject. The amplitude of the body sway may be conveyed by increasing the amplitude of the vibrators oscillations. As the vibrators will also set up a local bone-conducted oscillation of the skull, it follows from the aforementioned auditory feedback mode of the present invention, that a bone-conducted auditory tone will also be perceived by the hearing sense of the ear when the vibro-tactile feedback is activated, and vice versa for the bone-conducted auditory feedback mode, depending on how far away from the bone next to the ears the vibro-tactile transducer is placed. The threshold value, that is, the sway angle at which the vibro-tactile signal is first sensed either tactilely or auditory, the depth of vibro-tactile modulation with increasing sway, and the angle of sway in a direction of a particular vibrator that the modulation ceases to increase (or is limited), as well as if vibrators for a particular direction are active, are all vibro-tactile feedback parameters which may be adjusted by the operator to help improve the subject's control of body sway, and therefore improve the subject's balance control for one or more movement tasks.

Because it is well-known that vibration of the skull with a bone-conducting auditory transducer operating at a hearing level greater than 30 dB sensory level (where 0 dB represents the threshold of hearing for the subject), causes a sense of motion by activating vestibular receptors in the inner ear, it follows that vibration of the head whether it causes an auditory and/or vibro-tactile sensation will lead to excitation of vestibular receptors. Thus such excitation can also be used to convey a sense of body sway. The amplitude of such vibro-vestibular feedback may be conveyed by increasing the amplitude of the bone-conducting and vibro-tactile feedback once these have exceeded an auditory sensation level of 30 dB sensory level. By recording potentials in neck muscles in response to these stimuli it is possible to determine this threshold value individually and set this to a corresponding sway angle of the body. This sway angle at which the vibro-vestibular is first sensed, the depth of vibro-vestibular modulation with increasing sway, and the limit of sway when the vibro-vestibular modulation ceases to increase are all vibro-vestibular parameters which may be adjusted by the operator (albeit not independently of the acoustic and vibro-tactile parameters), to help improve the subject's control of body sway, and therefore improve the subject's balance control for one or more movement tasks.

A visual feedback system according to this invention may augment the above mentioned modes of feedback. For visual feedback, a system of 4 light emitting diodes (LED) are incorporated into a peak of the head-band worn by the subject. Such a system of LEDs will be so mounted only to be in the peripheral vision of the subject and not obstruct vision. The subject is thus able to see both the diodes and the world around him simultaneously. The visual feedback diodes are each of a different color to indicate sway in the left, right, backwards and forwards directions. Each diode flashes with an increasing frequency up to a frequency lower than the flicker frequency (when independent flashes can no longer be distinguished) with increasing body sway. Normally the threshold when the diodes begin to flash is larger than the thresholds for other forms of feedback in order to act as a fall warning. The sway angle when the diodes are first illuminated, the rate of increase in flash frequency with sway angle and the limit of sway when the flash frequency is held constant for further sway are all parameters which can be set and adjusted to help improve the subject's control of body sway, and therefore improve the subject's balance control for one or more movement tasks.

### BRIEF DESCRIPTION OF THE DRAWINGS

FIG. 1 is a schematic block diagram of an angular position and velocity based sway feedback and rehabilitation system in accordance with the present invention.

FIG. 2 is a schematic illustration of a human subject wearing body sway sensors that are used to provide body sway and angular velocity data in the roll and pitch directions in accordance with the present invention. A cable to a personal computer (PC) or microprocessor for collecting data may be present as shown, or the communication to the PC may be wireless.

FIG. 3 is an illustration of an exemplary operator's display for providing body sway angle and angular velocity information to an operator in accordance with the present invention.

FIGS. 4A, B contain schematic illustrations of a human subject wearing a device mounted at the head and capable of providing combined auditory bone-conducted, vibro-tactile, vibro-vestibular, and, in addition visual feedback of body sway and/or angular velocity information to the subject in accordance with the present invention.

FIG. 5 is a flow chart illustrating the steps of an exemplary system processor control program algorithm for sampling body sway sensors, providing an operator's display, and providing combined auditory bone-conducted, vibro-tactile, vibro-vestibular, and, in addition visual feedback of body sway and angular velocity information in accordance with the current invention.

### DETAILED DESCRIPTION OF EMBODIMENTS OF THE INVENTION

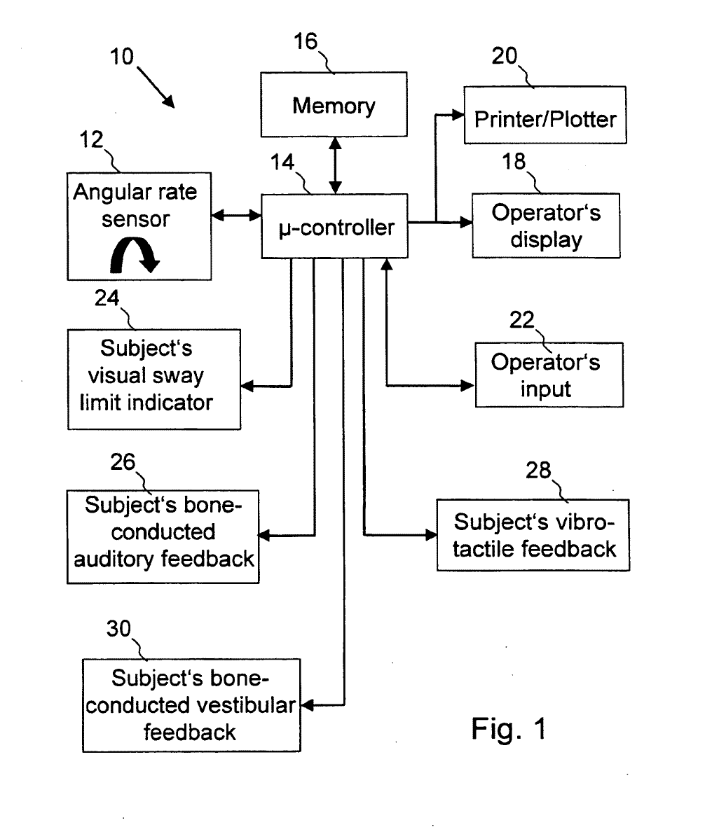

The present invention—inter alfa—provides a method and a system or apparatus for angular position and velocity based head-mounted multi-modal feedback system for the rehabilitation of balance and gait disorders by improving a subject's balance control during stance and gait. An angular position and velocity based rehabilitory system for monitoring body sway and providing body sway feedback to a subject to help improve the subject's control of body sway is illustrated generally at 10 in FIG. 1. Lightweight wearable body sway sensors 12 are attached to the lower back of a subject and provide body sway signals to a system processor 14 which derives body sway angle and angular velocity information therefrom. As will be discussed in more detail below, various types of body sway sensors, including angular velocity transducers, may be used to provide body sway signals, indicative of the trunk motion of a subject, to the system processor 14.

The system processor 14 may be implemented as a conventional microprocessor based computer system, commonly known as a personal computer or PC, or may be implemented a series of slave microprocessors connected to one or more master systems, either implementation having computer memory 16, an operator's display unit such as a standard computer display console 18, a printer/plotter 20, an input device 22, such as a conventional computer keyboard. The processor memory 16, may include short- and long-term memory. The microprocessor(s) 14, and memory 16 operates in a conventional manner to store the programmed series of instructions and algorithms that control operation of the microprocessor(s) 14 and to store data generated by the system processors.

In accordance with the present invention, the system processor 14 is programmed to transform the body sway signals provided by the body sway sensors 12 into useful body sway angle and body sway angular velocity information formats. This information is displayed by the system processor 14 in the form of an operator's display that is presented to the operator on the operator's display unit 18. From this formatted information, the system operator e.g. a physician is able to analyze the body sway of a subject during the performance of various motor tasks, to thereby assess whether there has been an improvement in the subject's performance when feedback of body sway angle and angular velocity information is provided to the subject in accordance with the present invention. A hard copy of the body sway information with and without feedback provided on the operator's display unit 18 may be obtained using the system printer/plotter 20. It should be noted that body sway sensors 12 may be attached to other portions of the subject's body, such as the head, upper or lower leg to provide information on the motion of the body segments the system processor 14.

In accordance with present invention, the system processor 14 is programmed to provide feedback of body sway angle and angular velocity information to a subject. As will be discussed in more detail below, this feedback may be provided to a subject in a combined bone-conducting auditory, vibro-tactile or vibro-vestibular from, and, in addition, a visual form. Visual 24, bone conducting auditory 26, vibro-tactile 28, and vibro-vestibular 30, feedback systems may, therefore, be provided in accordance with the present invention to deliver body sway angle and angular velocity feedback signals provided by the system processor 14 to the subject. It should be understood that multi-modal feedback consisting of a combination of bone-conducted auditory, vibro-tactile and vibro-vestibular is always provided to a subject unless the subject has a lack of function in two of these modes which is highly unlikely. An additional visual feedback may be provided too. It should be further understood that vibro-vestibular feedback will result non-independently from bone-conducting auditory or vibro-tactile feedback or a combination of the two and likewise bone-conducting auditory feedback will result in excitation of the other modes depending on transducer placement and stimulation intensity. Thus an angular position and velocity based body sway rehabilitory system 10 in accordance with the present invention need not include every feedback system 26, 28 and 30 illustrated in FIG. 1 but will include at least one of the feedback systems 26, 28 and 30 or any combinations of these three modes as a multi-modal feedback system.

It should be noted that the functions performed by the system processor 14 may be divided among several microprocessor based systems at different locations, and in communication either through direct physical connection e.g. using an RS-458 or 232 serial line interface or by wireless data transmission, e.g. using a Bluetooth™ communication system. For example a system processor attached to the subject's back may provide body sway angle and angular velocity information to another remote processor that provides the operator display, but also may be programmed to provide another processor mounted on the head at the location of the feedback transducers information on body sway angle and angular velocity to a feedback systems 24, 26, 28 and 30.

The location of the body sway sensors 12, 12B, 12C on the subject's body is determined by the axis of sensitivity of the sensors. The body sway sensors 12, 12B, 12C are preferably attached to the lower back of the subject using an elasticated belt 36 to thereby register at least the roll (side-to-side) 12B′ and pitch (forward-backward) motion 12C′ of the subject's body near to its centre of gravity.

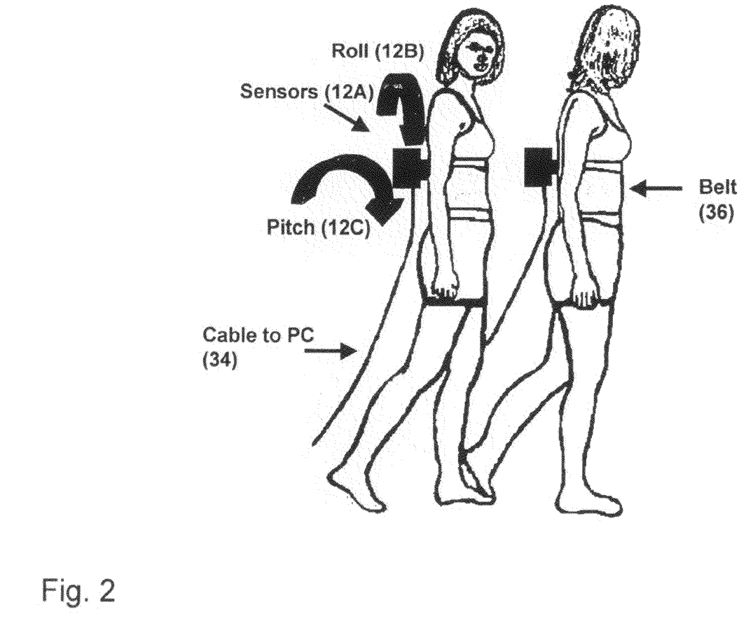

Preferable locations for the body sway sensors are illustrated in FIG. 2. Both sensors 12, 12B, 12C are mounted in a box 12A firmly attached to the belt 36 with a first sensor 12B within the box 12A mounted so as to register roll 12B′, that is, side-to-side angular deviations of the trunk from vertical, and roll angular velocities. A second sensor 12C is mounted in the sensor box 12A orthogonally so as to register pitch 12C′, that is, backward and forward angular deviations from the vertical, and pitch angular velocities. The sensors 12, 12B, 12C may be preferably secured to the lower back of subject by placing the sensors 12, 12B, 12C in a box 12A firmly attached to a hard plastic backing to the elastic belt 36 which may be of different sizes according to the waist circumference of the subject. It should be apparent, however, that other conventional means may also be used to attach the sensors 12B and 12C to the subject, either over or under the subject's clothing.

Various different types of sensors 12, 12B, 12C may be used to measure body sway angle and body sway angular velocity of the subject. Preferably the type of sensor that is used is capable of providing a direct measurement of angular velocity as described in U.S. Pat. No. 5,919,149, that is, an angular velocity sensor which is substantially insensitive to the gravity and to linear accelerations in each of the 3 dimensions (up and down, side-to-side, and backward-forward) of the subject's body at the location of the sensor box 12A. An exemplary and preferred body sway sensor is the Litef Micro Fors 36 fiber optic gyroscopic sensor made by Litef GmbH of Freiburg i. Br. Germany.

The body sway sensors 12, 12B, 12C may also be implemented using pairs of linear acceleration transducers (accelerometers) set at fixed distances from one another on a subject's body. Such devices may be used to measure angular accelerations, which may be transformed into angular velocity or angular deviation values by suitable digital integration algorithms implemented, for example, in the system processor 14. However, it is noted that most linear accelerometers have inherent drift problems that may be partially corrected using a compass reading. Thus the use of linear accelerometers to provide body sway sensing is not preferred, unless the drift problems are reduced by methods other than compass settings which are affected by artificially generated magnetic fields.

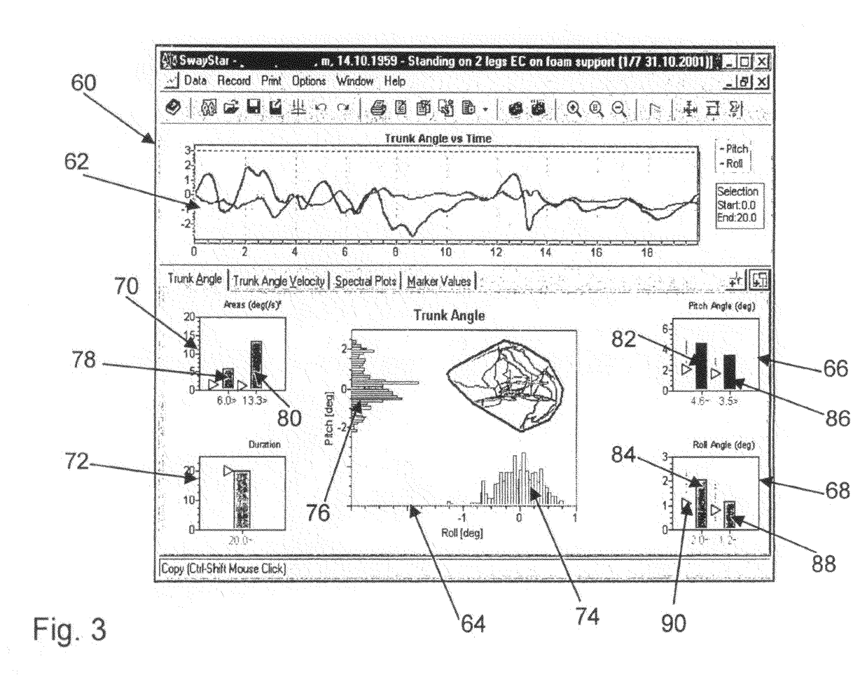

Transferred sensory signal information quantifying body sway angle and angular velocity may be displayed in a number of useful formats to an operator in order to enable the operator to determine an improvement in balance control with the multi-modal feedback operating according to the present invention. Such formats will be provided on the operator's display device 18. An exemplary preferred format for providing a display of a measured subject's trunk angular and angular velocity displacement is illustrated in FIG. 3. The preferred operator display 60 includes insert displays 62, 64, 66, 68 and 70 for displaying trunk angular displacements and angular velocity information in various formats. A time history of the subject's angular sway deviations in the pitch and roll directions over a selected part of the examination trial is provided in the top insert display 62. An x-y plot of the deviations in the roll, or x-direction, and pitch, or y direction, are plotted in the centre insert 64. This insert also preferably includes histograms of the body sway angle (or angular velocity) in the roll 74 and pitch 76 directions. A bar graph on the operator display indicates the total area circumscribed by the x-y plot (or its convex hull) for angular displacement 78 and angular velocity 80 deviations within the insert display 70. Bar graphs within the insert displays 66 and 68 provide an indication of the maximum and 90% range of instability of the subject in the roll and pitch directions, respectively. Maximum instability in either angular displacement or angular velocity is defined as the peak-to-peak displacement in the pitch and roll direction and is shown by the columns 82, 84, respectively. The 90% range of instability is defined within respect to the histograms 74, 76, and shown by bar graphs for pitch 86 and roll 88. Finally insert display 72 shows a bar graph of the time the subject took to complete the examination. The information provided in the bar graphs 78, 80, 82, 84, 86 and 88 may be displayed numerically as shown below the bar graphs in FIG. 3. Each piece of information provided in the operator display 60 is preferably updated at a rate of at least 1 time per second during the examination trial, which may typically last 5-30 seconds.

At the end of the examination trial the different types of information that are provided on the operator display 60 may be compared by the operator with previous values obtained from similar tests performed by the subject with and without the multi-modal feedback system according to the current invention, and with such values which represent those of a normal population not using feedback. The format for comparing with a normal reference population 90 consist of a median value represented by triangle apex in the insert display 68 plus a vertical bar showing the limits of the 5% and 95% values for a normal reference population whose ages lie within 5 years of that of the test subject.

As described above the present invention provides for rehabilitation of balance and gait deficits by the provision of body sway angle and angular velocity feedback to a subject. Such feedback is head-mounted and is in the form of a combined multi-modal feedback consisting of bone-conducted auditory, vibro-vestibular and in addition, in the form of visual feedback.

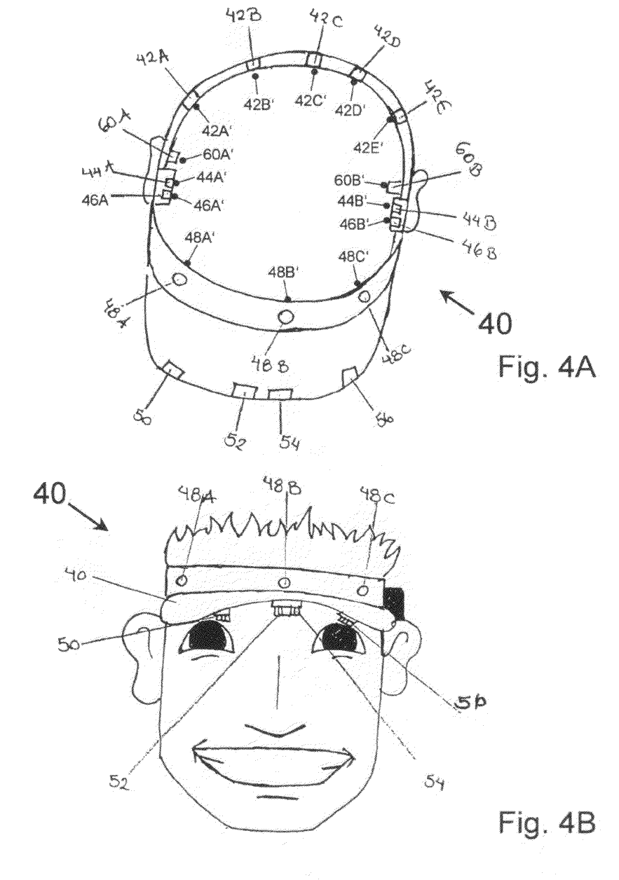

A system 40 for providing sway angle and angular velocity feedback is described with reference to FIG. 4.

The body sway information can, after suitable transformation, be presented to the subject aurally using bone-conducting vibrators 44A, 44B mounted into a head-band 40 so as to be placed just above the right 44A′ and left ears 44B′.

In the preferred embodiment of a bone-conducting auditory feedback system 26 of the inventive system 10, 40, pitch angular displacements backwards are presented as a tone amplitude modulation at a frequency of 1200 Hz at both the left and right bone-conducting vibrators, and pitch angular displacements forwards are presented as a tone amplitude modulation at a centre frequency of 300 Hz at both the left and right bone-conducting vibrators 44A and 44B. Increased sway amplitude or amplitude of sway angular velocity may be presented as an increase in the tone amplitude at both left and right vibrators 44A and 44B. Roll angular displacements to the left are presented as a tone amplitude modulation at a centre frequency of 500 Hz with a small frequency modulation every 20 ms some 5% of 500 Hz, at the left bone-conducting vibrator 44B. Roll angular displacements to the right are presented as a tone amplitude modulation at a centre frequency of 800 Hz with a small frequency modulation at every 20 ms some 5% of 800 Hz. Increases in sway angular amplitude or velocity to the left or right are represented at the left and right bone-vibrators, respectively as an increase in the amplitude modulation of the tones. An exemplary and preferred bone-conducting vibrator is the Bone-Conductor KH20-11 made by BHM-Tech Produktionsgesellschaft mbH, Austria and having a band width of auditory stimulation between 300 and 2500 Hz. For the bone-conducting auditory feedback system 26, the amplitude of sway at which a tone is first heard, the amplitude of sway at which the tone amplitude is limited, the gain between threshold and the maximum limit and directions of sway for which no tone is heard, are preferable variable parameters of the bone-conducting auditory feedback gain. The feedback parameters may be set by the operator to help improve the subject's control of sway and therefore improve the subject's balance control for one or more movement tasks.

For vibro-tactile feedback 28 of the system 10, 40, vibrators 46A, 46B, 60A, 60B and 42A, 42B, 42C, 42D, 42E and may be used to provide body sway angle and angular velocity feedback to the subject. Several vibrators 46A, 46B, 60A, 60B and 42A, 42B, 42C, 42D, 42E may be placed at the side of the head 46A′, 46B′, 60A′, 60B′ around the back of the head 42A′, 42B′, 42C′, 42D′, 42E′ where sensitivity of the skin is greater than at the front of the head, and/or around the front of the head 48A′, 48B′, 48C′. When the subject sways in a direction defined by one of the vibrators 42A-D, 46A-B and 48A-C, 60A-B and in segments around that direction, the associated vibrator will start oscillating and create a tactile sensation at the skin and therefore perceived by the brain. The angle of sway at which the vibrator switches on may be defined by the operator as one of the feedback gain parameters. An exemplary and preferred number of vibrators is 3 at the front of the head (48A-C), one at each side of the head (46A and 46B), and 3 at the back of the head (42A, 42C, and 42E). From the foregoing it is clear that the operator may switch off the vibro-tactile vibrators for some directions of sway in order to enhance sway control in non-switched off directions as one of the feedback gain parameters. An increase in the amplitude of sway up to a certain limit may be sensed by the subject as an increased amplitude modulation. An exemplary and preferred type of vibro-tactile vibrator is the C1234L-38 produced by Vibrator Motor. Com runs at ca 200 Hz. The above mentioned feedback parameters may be set by the operator to help improve the subject's control of sway, and therefore improve the subject's balance control for one or more movement tasks.

Body sway angle and angular velocity may also be provided in the form of bone-conducted vibration of the skull that excites vestibular receptors in the inner ear of the subject. Such a feedback system 30 of the inventive system 10, 40 will be provided by a combination of the bone-conducted auditory feedback signals and vibro-tactile signals that will lead to a verifiable vestibular response of the subject's brain depending on the location and intensity of skull vibration. One means of verifying the presence of such signals is to provide a 500 Hz tone burst of duration 6 ms as signals to the auditory 26 and vibro-tactile 28 feedback systems and observe the subsequent responses in pre-activated neck muscles. The combination of auditory 26 and vibro-tactile 28 feedback signals that then provides an observable response can be used as one parameter of the bone-conducted vestibular feedback system 30 when this is associated with a particular direction and amplitude of sway. Increases in the amplitudes of the auditory and vibro-tactile system vibrators with increases in body sway angle, as well as whether the feedback is present for other sway directions are preferably variable parameters that may be set by the operator to help improve the subject's balance control for one or more movement tasks.

A system 24 of the inventive system 10, 40 for providing visual body angle and angular velocity feedback in addition to the multi-modal feedback systems 26, 28 and 30 includes four (4) light emitting diodes (LEDs) 50, 52, 54, 56 mounted as shown in FIG. 4 in the peak of the head-band worn by the subject. The manner and color of illumination of the LEDs provides visual feedback to the subject of his body sway and angular velocity in the peripheral field of view without interfering with his vision. Together with the other feedback forms 26, 28 and 30 the subject may use this information to augment the balance signals normally used by his brain to correct body sway instabilities. A preferred format for providing the visual feedback of the subject's body sway angle and angular velocity is for the right-most LED 50 to be illuminated with a green color when a threshold value of body sway to the right set by the operator is reached, for the left-most LED 56 to be illuminated blue for left sway when a threshold value is reached, and for the two frontal LEDs 52 and 54 to be illuminated red and yellow when backward and forward sway angle thresholds are exceeded, respectively. Further increases in sway angle and/or sway velocity are preferably feedback to the subject by an increasingly greater flicker frequency of the LED depending on the direction of sway until a limit set by the operator at which a fall could occur because the subject exceeded a cone of stability. The term “cone of stability” may be defined as the maximum leaning position of the upper body, that is, the trunk, that a subject can achieve standing for two seconds, without falling, while attempting to keep his body as straight as possible. A normal cone of stability may be defined is that average set of values achieved by a group of at least 12 normal subjects that are within 5 years of age of the test subject. Another definition of the cone of stability is to make it equal to the maximum lean of the trunk for a normal group of subjects when they performed a specific task such as standing eyes closed on a foam support surface. Whichever definition is used, when the values of the cone of stability are exceeded a fall warning will be issued, and a percentage of the cone of stability, such as 10%, taken as the threshold value when visual feedback (or other types of feedback) is first active. The cone of stability may be, for example, in eight radial directions extending from the axis of the subject's upper body (that is forward, forward right, right, backward right, backward, backward left, left and forward left). The amount of sway for a fall warning, for a threshold value for feedback, and for a limit of a feedback increase may be set as the amount of sway in each of these 8 directions or more than or less than 8 directions but not less than 4 directions. These feedback parameters, together with the gain of the visual feedback (increase in flicker frequency per deg sway) may be set by the operator to help improve the subject's balance control for one or more movement tasks. The visual gain parameters, like those of the other feedback systems 26, 28 and 30 may be fine tuned by repeated movement task examination trials, for example with the aim of reducing the thresholds within which the subject attempts to maintain sway.

The multi-modal feedback system described herein may also be used to provide feedback of angular deviations and/or angular velocities of parts of the subject's body other than the trunk. This requires, however, that body motion sensors be provided at the appropriate other locations on the subject's body.

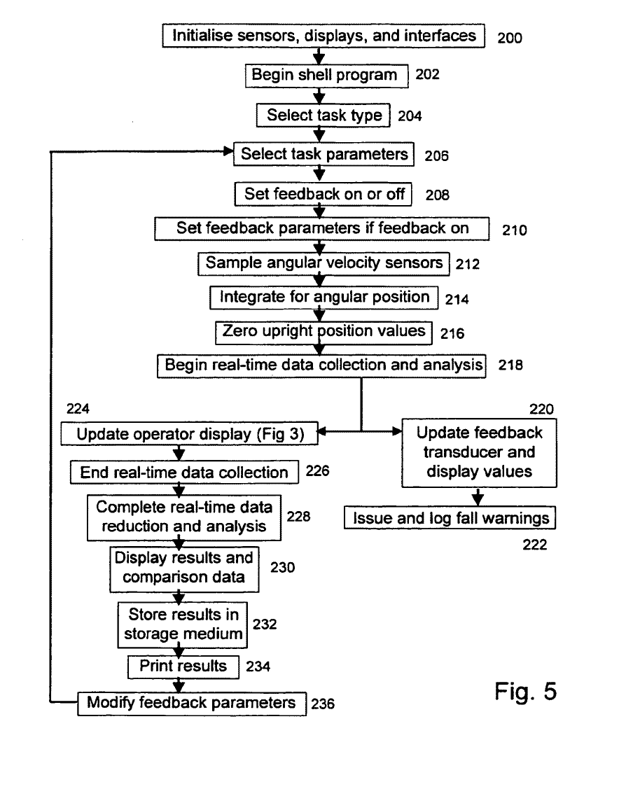

A flow chart illustrating an exemplary procedure that may be implemented by the system processor 14 to control the flow of information provided by the sensors 12, to generate the operator's display 60, to control the different feedback systems 24, 26, 28 and 30, and to allow an operator to intervene in the flow of information using an operator's input device 22 is described with reference to FIG. 5. It should be apparent that the steps of this procedure may be implemented as a computer program for controlling operation of the system processor using conventional programming techniques. The first step 200 that the processor 14 completes prior to enabling operator control is to initialize and verify communication between the processor 14, the body sway sensors 12, the operator's display unit 18, the feedback systems 24, 26, 28 and 30, and any other interfaces. At step 202 a shell program is entered which allows an operator to enter subject data such as name, date of birth, sex and to enter visit specific data such as the medical history, weight and height. At step 204 the operator is able to select the task type which the subject is to perform during the examination trial, together with the display features for the operator's display such as maximal duration, and scaling of traces for time histories of the measurement variables. At step 206 the operator is able to select the feedback gain, threshold and limit parameters from a list of preselected values or enter new feedback parameters. At step 208 the feedback systems among 24, 26, 28 and 30 are set on or off and in step 210 these feedback parameters are communicated to the microprocessors controlling the feedback systems. At step 212 the angular velocity transducers 12 are sampled prior to the start of the actual recording and integrated continuously in step 214 to yield angular position. When the operator issues a start command to begin the recording, the angular position values are initialized, that is set to zero position in step 216 and then realtime data collection at a preferred rate of 100 samples per second and online analysis is performed in step 218. As has been described, the present invention may provide body sway angle and angular velocity feedback to the subject to improve the subject's gait and balance control. For the feedback systems 24, 26, 28 and 30 this includes updating the feedback transducer and display values, which is performed a step 220 and issuing and logging any fall warnings to the subject as well as to the system operator in step 222. Simultaneously with updating the feedback values in step 220, the operator's display 18 (as shown in exemplary form in FIG. 3) is updated in step 224. It should be apparent that steps 218 to 224 are repeated continuously throughout the examination period. On completion of the data acquisition phase, at step 226, the data gathered during the examination trial period is analyzed, reduced, and redisplayed to the operator in the operator's display 60 as part of step 228. At step 230, the previously stored body sway data may be retrieved from the system memory 16 for comparison with the information obtained during the examination trial period. Such previously stored information may include information from normal or other sample populations for the particular task performed during the examination trial, and/or may be the particular subject's previous trial results for the current task with the feedback switch on or off. At step 232 the data from the current examination trial is stored in the processor system's memory 16. The operator may choose to print these results on the printer/plotter 20, at step 234. Based on the operator's examination of the examination trial results at step 230, the operator may change the feedback parameters at step 236, with the aim of further reducing the subject's trunk sway for the current task. The examination trial may then be repeated, by beginning real time data collection at step 218.

It should be apparent that the control procedures other than the one described herein for exemplary purposes may be employed in accordance with the current invention. The order of the procedural steps may be changed, steps may be added, and others may be deleted. Additionally some of these procedured steps may occur simultaneously on separate system processors. For example the step 220 of updating the multi-modal feedback may be implemented on one portable processor carried by the subject at the headband, whereas the step of integrating the angular velocity sample values from the transducer or sensor 12 may be implemented by a processor in the sensor box 12A, and the operator's display 60 may be updated by a third remote processor.

The present invention is further elucidated based on the following remarks:

A method and an apparatus or system for the rehabilitation of abnormal postural sway of a subject during standing or the performance of movement tasks using a multi-sensory-modal head-mounted system feedback are provided. For this purpose body sway sensors, such as angular velocity transducers or linear accelerometers, are attached to the body, such as the lower back, of subject. Output signals from the body sway sensors are transformed into detailed body sway angular displacement and velocity information by a system processor. The body sway angular displacement and velocity information may be displayed to the operator for controlling the efficacy in improving the amount of sway when a specific combination of angular displacement and velocity information is provided as multi-modal feedback to the subject. This multi-modal feedback may be augmented by visual feedback in the form of a head-mounted display. The feedback is provided to augment the signals normally used by the subject's brain to help stabilize body sway and improve balance. Rehabilitatory feedback consists of several head-mounted transducers which simultaneously can provide feedback in form of vibro-tactile sensation at the skin of the head, bone vibration of the skull causing an auditory sensation in the inner ear, and vestibular sensation by skull vibration exciting vestibular receptors in the inner ear. This multi-modal feedback may be augmented by visual feedback in the form of a head-mounted display. Each feedback mode may be programmed to commence stimulation at a specific threshold of body angular sway and not to increase stimulation intensity past a specific limit of body angular sway. An angular position and velocity based body sway system in accordance with the current invention may be used to provide rehabilitatory feedback to subjects without interfering with or restricting the normal movement activities or visual or auditory perception of the subject.

The present invention inter alia relates to a system 10 for providing body sway feedback to a body of a subject, comprising (a) at least one body sway sensor means 12 adapted to be attached the body and adapted for providing body sway signals indicative of a sway of the body, (b) a system processor means 14 adapted for deriving body sway information from the body sway signals and adapted for generating a body sway feedback signal from the body sway information, and (c) a subject body sway feedback means 40 having at least one transducer unit 42, 44 and adapted for providing a body sway feedback to the body via said at least one transducer unit 42, 44 based on said body sway feedback signal, (d) wherein said at least one transducer unit 42, 44 is or comprises at least one transducer, (e) wherein said at least one transducer is adapted to be contacted to a head of the body, adapted for vibrating, and adapted for transferring a respective vibration to the head, and (f) wherein said at least one transducer is adapted to evoke based on the vibration a multimodal body sway feedback as stimuli to the subject having a plurality of kinds of stimuli of the group comprising auditory stimuli, tactile stimuli, and vestibular stimuli.

Said at least one transducer 42, 44 may be adapted for transferring said respective vibration to the head via a process of skin and/or bone conduction.

Said at least one transducer 42, 44 may be adapted to evoke said multimodal body sway feedback based on vibration frequency and/or vibration amplitude.

The body sway sensor 12 may be adapted to be attached to the lower back of the subject's body.

The system may include a body sway sensor 12 adapted to be attached to the body of the subject to provide body sway signals indicative of the sway of the body in a pitch direction 12C′ and a body sway sensor 12 adapted to be attached to the body of the subject to provide body sway signals indicative of the sway of the body in a roll direction 12B′.

The body sway sensor 12 may include an angular velocity sensor providing body sway signals indicative of the angular velocity of the body.

The body sway sensor 12 may include acceleration sensors providing body sway signals indicative of the angular acceleration of the body.

The body sway sensor 12 may include a position sensor providing body sway signals indicative of the angular displacement of the body.

The system processor 14 means may include means for deriving body sway angle and body sway angular velocity information from the body sway signals and means 40 for generating the body sway feedback from the body sway angle and body sway angular velocity information.

The subject body sway feedback means 40 may include means 26 for providing the body sway feedback to the subject aurally via bone conduction to the hearing receptors of inner ear and is adapted to be via one or more bone vibrating transducers 42, 44 mounted around the circumference of the head.

The subject body sway feedback means 40 may also include means 28 for providing the body sway feedback to the subject through vibro-tactile stimulation of the subject's body and is adapted to be via one or more vibrating transducers 42, 44 mounted around the circumference of the head.

The subject body sway feedback means may also include means for providing the body sway feedback to the subject through vibro-vestibular stimulation of the subjects head and is adapted to be via one or more vibrating transducers 42, 44 mounted at the circumference of the head.

The subject body sway feedback means 40 may include means for providing the body sway feedback to the subject visually.

Said means for providing the body sway feedback visually may include a head mountable subject display means for displaying a subject feedback display to the subject.

The system processor means 14 may also include means for generating a subject feedback display including groups of light emitting diodes 50-56 wherein the diode light illuminated is of a different color when the body moves forward and backward, likewise when the body sways left and right.

The subject body sway feedback means 40 may include means for providing the body sway feedback to the subject through any combination of visual and aural, vibro-tactile, vibro-vestibular means.

The body sway feedback means 40 may use the same or different transducers 42, 44 mounted at the same point on the head to provide the vibration.

The body sway feedback may be related to the body sway information by feedback gain parameters, and comprising additionally means for adjusting the feedback gain parameters such as threshold, maximum limits, gains between the threshold and maximum limits, and the relative gain of angular position, velocity and acceleration feedback.

The system processor means 14 may include means for comparing the body sway information with a set of safety rules defining an angular cone of stability of the subject and for incorporating a fall warning indication into the body sway feedback when the subject's body sway exceeds an angle greater than a selected fraction of the angular cone of stability, and wherein the subject body sway feedback means includes means for providing the fall warning indication to the subject.

The method for providing body sway feedback to a body of a subject, comprises the steps (A) providing body sway signals indicative of a sway of the body, (B) deriving body sway information from the body sway signals and generating a body sway feedback signal from the body sway information, and (C) providing a body sway feedback to the body based on said body sway feedback signal, (D) wherein a vibration is provided as said body sway feedback, (E) wherein said vibration is transferred to a head of the body of the subject, and (F) wherein based on said vibration a multimodal body sway feedback is evoked as stimuli to the subject having a plurality of kinds of stimuli of the group comprising—auditory stimuli,—tactile stimuli, and—vestibular stimuli.

Said vibration may be transferred to the head of the body of the subject via a process of skin and/or bone conduction.

Said a multimodal body sway feedback may be evoked based on vibration frequency and/or vibration amplitude.

A body sway sensor 12 may be used which is attached to the lower back of the subject's body.

A step of attaching a body sway sensor 12 to the body of a subject may include the steps of attaching a first body sway sensor 12C to the body of the subject to provide body sway signals indicative of the sway of the body in a pitch direction 12C′ and attaching a second body sway sensor 12B to the body of the subject to provide body sway signals indicative of the sway of the body in a roll direction 12B′ or attaching both sensors 12, 12B, 12C together.

A body sway sensor 12 may be used which includes an angular velocity sensor providing body sway signals indicative of the angular velocity of the body.

A body sway sensor 12 may be used which includes acceleration sensors providing body sway signals indicative of the angular acceleration of the body.

A body sway sensor 12 may be used which includes a position sensor providing body sway signals indicative of the angular displacement of the body.

A step of generating an operator display may include a step of generating a time history of the subject's body sway angle and body sway angular velocity over a trial period.

The method may comprise additionally steps of comparing the body sway angle information with a set of safety rules defining a cone of stability of the subject to obtain a measure of the subject's balance control and for incorporating the measure of the subject's balance control in the operator display.

The method may comprise a step of comparing the body sway angle and angular velocity information with and without feedback to normal body sway information.

The method may comprise a step of comparing the body sway angle and angular velocity information to body sway information of the subject obtained during past performance of a particular task with and without feedback.

The step of providing the body sway feedback to the subject may include the step of providing the body sway feedback to the subject aurally and is adapted to be via one or more bone vibrating transducers 42, 44 mounted around the circumference of the head.

The step of providing the body sway feedback to the subject may include additionally the step of providing the body sway feedback to the subject through vibro-tactile stimulation of the subject's body and is adapted to be via one or more vibrators 42, 44 mounted around the circumference of the head.

The step of providing the body sway feedback to the subject may also include the step of providing the body sway feedback to the subject through vibro-vestibular stimulation of the subject's body and is adapted to be via one or more vibrators 42, 44 mounted around the circumference of the head.

The body sway method may use the same or different transducers 42, 44 mounted at the same point on the head to provide the vibration.

The method may comprise steps of comparing the body sway angle information with a set of safety rules defining an angular cone of stability of the subject, generating a fall warning indication when the subject's body sway exceeds an angle greater than a selected fraction of the angular cone of stability, and providing the fall warning indication to the subject.

According to another view of the present invention a system is proposed for providing augmenting prosthetic body sway feedback in multi-modal form to improve a subject's balance and movement control, comprising (a) a body sway sensor adapted to be attached the body of a subject and providing body sway signals indicative of the sway of the body; (b) a system processor means for deriving body sway information from the body sway signals and for generating body sway feedback from the body sway information; and (c) a subject body sway feedback means for providing multi-modal body sway feedback to the subject via one or more head-mounted vibrating transducer units.

According to another view of the present invention a method is proposed for monitoring the body sway of a subject with and without feedback of sway to the subject, comprising the steps of (a) attaching a body sway sensor to the body of a subject, the body sway sensor providing body sway signals indicative of the sway of the body; (b) deriving body sway angle and body sway angular velocity information from the body sway signals and generating an operator display of the body sway angle and body sway angular velocity information; and (c) displaying the operator display to an operator of the system, (d) attaching to the body a multi-modal feedback means in the form of one or more head-mounted transducer units.

It should be understood that this invention is not confined or limited to the particular embodiments, implementations, and applications herein illustrated and described, but enhances all such modified forms thereof as come within the scope of the following claims:

### LIST OF REFERENCE SYMBOLS

10 system according to an embodiment of the present invention
12 sensor
12A box, sensor box
12B 1^{st} sensor, roll sensor
12B′ roll
12C 2^{nd} sensor, pitch sensor
12C′ pitch
14 system processor
16 memory
18 display, display unit
20 printer, plotter
22 input means, keyboard
24 visual feedback system
26 auditory feedback system
28 vibro-tactile feedback system
30 vibro-vestibular feedback system
40 body sway feedback means, head-band based body sway feedback device
42 transducer, transducer unit
42A-E transducer at the back of the head
42A′-E′ positions at the back of the head
44 transducer, transducer unit
44A transducer, bone-conducting vibrator
44A′ right ear, position thereof
44B transducer, bone-conducting vibrator
44B′ left ear, position thereof
46A, B transducer at position at the side of the head
46A′, B′ position at the side of the head
48A′-C′ position at the front of the head
50-56 LEDs
60 operator display
60A, B transducer at position at the side of the head
60A′, B′ position at the side of the head
62 display field, display insert
64 display field, display insert
66 display field, display insert for 90% range of instability in roll direction
68 display field, display insert for 90% range of instability in pitch direction
70 display field, display insert
74 display for body sway angle in roll direction
76 display for body sway angle in pitch direction
78 display for angular displacement
80 display for angular velocity
82 display for peak-to-peak displacement in pitch direction
84 display for peak-to-peak displacement in roll direction
86 90% range of instability in pitch direction
88 90% range of instability in pitch direction
90 comparison to normal reference values

1-36. (canceled)

37. A system for providing multi-modal body sway feedback to a body of a subject, comprising:
(a) at least one body sway sensor capable of being attached at a lower back of the body and providing body sway signals indicative of a sway of the body, the at least one body sway sensor comprising at least one of:
an angular velocity sensor capable of providing body sway signals indicative of an angular velocity of the body;
an acceleration sensor system capable of providing body sway signals indicative of an angular acceleration of the body; and
a position sensor capable of providing body sway signals indicative of an angular displacement of the body;
(b) a system processor capable of deriving body sway information from the body sway signals and capable of generating a body sway feedback signal from the body sway information;
(c) a subject body sway feedback device having at least first and second transducer units and capable of providing a body sway feedback to the body through the at least first and second transducer units based upon the body sway feedback signal;
(d) the first transducer unit:
capable of being removably secured to a head of the body; and
capable of applying a relatively low-level vibration and transferring the low-level vibration to skin of the head; and
(e) the second transducer:
capable of applying a relatively high-level vibration; and
capable to evoke, with the first transducer, a multimodal body sway feedback as stimuli to the subject, the stimuli selected from at least one of the group consisting of:
auditory stimuli;
tactile stimuli; and
vestibular stimuli,
based upon the high-level vibration of a bone of a skull of the body, the high-level vibration being insufficient to cause eye movements through the vestibular ocular-reflex.

38. The system according to claim 37, wherein the at least one body sway sensor is capable of providing body sway signals indicative of the sway of the body in a pitch direction and is capable of providing body sway signals indicative of the sway of the body in a roll direction.

39. The system according to claim 37, wherein the system processor comprises:
a derivation device capable of deriving body sway angle and body sway angular velocity information from the body sway signals; and
feedback generating device capable of generating the body sway feedback from the body sway angle and body sway angular velocity information.

40. The system according to claim 37, wherein the system processor comprises:
means for deriving body sway angle and body sway angular velocity information from the body sway signals; and
means for generating the body sway feedback from the body sway angle and body sway angular velocity information.

41. The system according to claim 37, wherein the subject body sway feedback device comprises a feedback device capable of providing the body sway feedback to the subject aurally through bone conduction to hearing receptors of an inner ear through at least one bone vibrating transducer mounted just above ears of the head.

42. The system according to claim 37, wherein the subject body sway feedback device comprises means for providing the body sway feedback to the subject aurally through bone conduction to hearing receptors of an inner ear through at least one bone vibrating transducer mounted just above ears of the head.

43. The system according to claim 37, wherein the subject body sway feedback device further comprises a feedback device for providing the body sway feedback to the subject through vibro-tactile stimulation of the body through at least one vibrating transducer mounted around a circumference of the head.

44. The system according to claim 37, wherein the subject body sway feedback device further comprises means for providing the body sway feedback to the subject through vibro-tactile stimulation of the body through at least one vibrating transducer mounted around a circumference of the head.

45. The system according to claim 37, wherein the subject body sway feedback device further comprises a feedback device capable of providing the body sway feedback to the subject through vibro-vestibular stimulation of the head through at least one vibrating transducer mounted just above ears of the head, the stimulation being insufficient to cause eye movements through the vestibular ocular-reflex.

46. The system according to claim 37, wherein the subject body sway feedback device further comprises means for providing the body sway feedback to the subject through vibro-vestibular stimulation of the head through at least one vibrating transducer mounted just above ears of the head, the stimulation being insufficient to cause eye movements through the vestibular ocular-reflex.

47. The system according to claim 37, wherein the subject body sway feedback device is capable of providing the body sway feedback to the subject through at least one of the group consisting of aural feedback, vibro-tactile feedback, and vibro-vestibular feedback.

48. The system according to claim 37, wherein the subject body sway feedback device includes means for providing the body sway feedback to the subject through at least one of the group consisting of aural feedback, vibro-tactile feedback, and vibro-vestibular feedback.

49. The system according to claim 37, wherein the body sway feedback is related to the body sway information by feedback gain parameters selected from at least one of the group consisting of threshold, maximum limits, gains between the threshold and the maximum limits, and a relative gain of angular position, velocity and acceleration, and which further comprises an adjustment device capable of adjusting the feedback gain parameters between the at least first and second transducers.

50. The system according to claim 37, wherein the body sway feedback is related to the body sway information by feedback gain parameters selected from at least one of the group consisting of threshold, maximum limits, gains between the threshold and the maximum limits, and a relative gain of angular position, velocity and acceleration, and which further comprises additionally means for adjusting the feedback gain parameters between the at least first and second transducers.

51. A system for providing multi-modal body sway feedback to a body of a subject, comprising:
(a) at least one body sway sensor means capable of being attached at a lower back of the body and providing body sway signals indicative of a sway of the body, the at least one body sway sensor comprising at least one of:
an angular velocity sensor capable of providing body sway signals indicative of an angular velocity of the body;
an acceleration sensor system capable of providing body sway signals indicative of an angular acceleration of the body; and
a position sensor capable of providing body sway signals indicative of an angular displacement of the body;
(b) a system processing means capable of deriving body sway information from the body sway signals and capable of generating a body sway feedback signal from the body sway information;
(c) a subject body sway feedback means having at least first and second transducer units and capable of providing a body sway feedback to the body through the at least first and second transducer units based upon the body sway feedback signal;
(d) the first transducer unit:
capable of being removably secured to a head of the body; and
capable of applying a relatively low-level vibration and transferring the low-level vibration to skin of the head; and
(e) the second transducer:
capable of applying a relatively high-level vibration; and
capable to evoke, with the first transducer, a multimodal body sway feedback as stimuli to the subject, the stimuli selected from at least one of the group consisting of:
auditory stimuli;
tactile stimuli; and
vestibular stimuli,
based upon the high-level vibration of a bone of a skull of the body, the high-level vibration being insufficient to cause eye movements through the vestibular ocular-reflex.

52. A method for providing body sway feedback to a body of a subject, which comprises:
providing body sway signals with at least one of:
an angular velocity sensor providing body sway signals indicative of an angular velocity of the body;
an angular acceleration sensor system providing body sway signals indicative of an angular acceleration of the body; and
a position sensor providing body sway signals indicative of an angular displacement of the body;
deriving body sway information from the body sway signals and generating a body sway feedback signal from the body sway information;
providing a body sway feedback in the form of a vibration to the body based upon the body sway feedback signal and transferring the vibration to a head of the body; and
evoking a multimodal body sway feedback based upon the vibration as stimuli to the subject, the stimuli being selected from at least one of the group consisting of:
auditory stimuli;
tactile stimuli; and
vestibular stimuli.

53. The method according to claim 52, which further comprises carrying out the vibration transfer to the head through at least one of skin conduction and bone conduction.

54. The method according to claim 52, which further comprises carrying out the evoking step based upon at least one of a vibration frequency and a vibration amplitude.

55. The method according to claim 52, which further comprises providing the body sway signals by attaching a body sway sensor to a lower back of the subject, the body sway sensor having at least one of:
a first body sway sensor capable of providing body sway signals indicative of the sway of the body in a pitch direction; and
a second body sway sensor capable of providing body sway signals indicative of the sway of the body in a roll direction.

56. The method according to claim 52, which further comprises comparing the body sway angle and body sway angular velocity information of the body with and without feedback by generating an operator display including a time history of the body sway angle and body sway angular velocity over a trial period with and without feedback.

57. The method according to claim 52, wherein the step of providing the body sway feedback to the subject is carried out by at least one of:
providing the body sway feedback to the subject aurally through at least one vibrating transducer mounted just above ears of the body;
providing the body sway feedback to the subject through vibro-tactile stimulation of the body through at least one vibrator mounted around a circumference of the head; and
providing the body sway feedback to the subject through vibro-vestibular stimulation of the body through at least one vibrator mounted just above the ears, the stimulation being at a level insufficient to cause eye movements through the vestibular ocular-reflex.
